(19) United States
(12) Patent Application Publication
(10) Pub. No.: US 2009/0074609 A1
(43) Pub. Date: Mar. 19, 2009

(54) Apparatus for Odour Removal From an Input Gas

(76) Inventor: Raymond Thomas Malyon, Surrey (GB)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 11/886,028

(22) PCT Filed: Mar. 10, 2006
(86) PCT No.: PCT/GB2006/000888

§ 371 Date: Dec. 1, 2008

(30) Foreign Application Priority Data
Mar. 10, 2005 (GB) 0504916.8

(51) Int. Cl. A61L 9/015; A61L 9/20

(52) U.S. Cl. 422/4; 422/123; 422/121

(57) ABSTRACT

An apparatus for removing unpleasant odour by means of a first section (10) through which the input gas is constrained to travel and which treats the input gas by exposure to ozone and a second section (11) which converts into oxygen any remaining ozone in the gas stream issuing from the first section. The first and second sections are spaced apart by an interconnecting duct. The ozone is produced from air in the input gas, or alternatively the ozone is produced from air outside the input gas and introduced into the input gas in the first section.

### CROSS-REFERENCE TO RELATED APPLICATION

This is a §371 application of PCT/GB2006/000888 filed Mar. 10, 2006, which claims the benefit of Great Britain Application No. 0504916.8 filed on Mar. 10, 2005, both of which are incorporated herein by reference.

### TECHNICAL FIELD

The present invention relates to the removal of unpleasant odours, grease, or other contaminants from an input gas.

In the past, removal of odours from input gas has often required the use of charcoal filters and while this is an effective technique in many circumstances, it is not suitable for use in all circumstances.

### DISCLOSURE OF INVENTION

It is an object of the present invention to provide apparatus which will remove unpleasant odours from an input gas stream without the use of odour absorbing filters or any charged plates. In particular, it is an object of the invention to provide apparatus for removing odours and grease particles from the gas stream entering an extractor hood, such as that used in a commercial or domestic kitchen.

The present invention provides an apparatus for removing unpleasant odours by means of a first section through which the input air is constrained to travel and which treats the input gas by exposure to ozone and a second section which converts into oxygen any remaining ozone in the gas stream issuing from the first section, wherein the first section is spaced apart from the second section.

The first section and the second section are separated by an interconnecting duct, which provides additional time during the transit of the gas stream for the treatment of the gas by the ozone, in particular the breaking down of grease particles, before any remaining ozone is removed in the second section.

The first section may itself include means for producing ozone from air in the input gas, or alternatively the apparatus may include means for producing ozone from air outside the input gas, and introducing the produced ozone into the input gas in the first section.

Ultraviolet light is used in order to create the ozone and ultraviolet light at a different wavelength to the first mentioned ultraviolet is utilised to convert ozone to oxygen in the second section.

The ultraviolet light used to produce the ozone is at 185 nanometers and the ultraviolet light in the second section is at 254 nanometers wavelength.

A further aspect of the invention provides a method for removing unpleasant odours from an input gas, the method comprising the steps of:

providing first gas processing means through which the input gas is constrained to travel and which treats the input gas by means of exposing the input gas to ozone;

providing second gas processing means which converts into oxygen any remaining ozone in the air stream issuing from the first gas processing means; and

locating the first gas processing means at an inlet of a section of duct and the second gas processing means at an outlet of the section of duct so that the first and second gas processing means are spaced apart along the duct.

The first gas processing means treats the input gas by producing ozone from air in the input gas. Alternatively, the method comprises the step of producing ozone from air outside the input gas, and introducing the produced ozone into the input gas in the first gas processing means, thereby allowing the ozone producing means not to be exposed to the contaminants in the input gas.

### BRIEF DESCRIPTION OF THE DRAWINGS

In order that the present invention be more readily understood, embodiments thereof will now be described with reference to the accompanying drawings in which:

FIG. 1 shows a diagrammatic cross-section through the first section of the apparatus according to the present invention;

FIG. 2 shows a diagrammatic cross-section through the second section;

FIG. 3 shows an embodiment of the invention as part of a kitchen extractor system;

FIG. 4 shows an alternative embodiment of the invention as part of a kitchen extractor system;

FIGS. 5(a) and 5(b) respectively show different views of an ozone generator used in the embodiment of FIG. 4;

FIG. 6 shows schematically the operating principle of the ozone generator of FIGS. 5(a) and 5(b);

FIGS. 7(a) and 7(b) show in more detail the attachment of the ozone generator shown in the arrangement of FIG. 4;

FIGS. 8(a) and 8(b) show in more detail the ‘stab-in’ duct mounted UV lamp unit shown in the arrangement of FIG. 4;

FIG. 9 shows in more detail an arrangement for mounting a UV lamp unit in a kitchen extractor hood; and

FIG. 10 shows a modification of the apparatus shown in FIGS. 1 and 2.

### BEST MODE FOR CARRYING OUT THE INVENTION

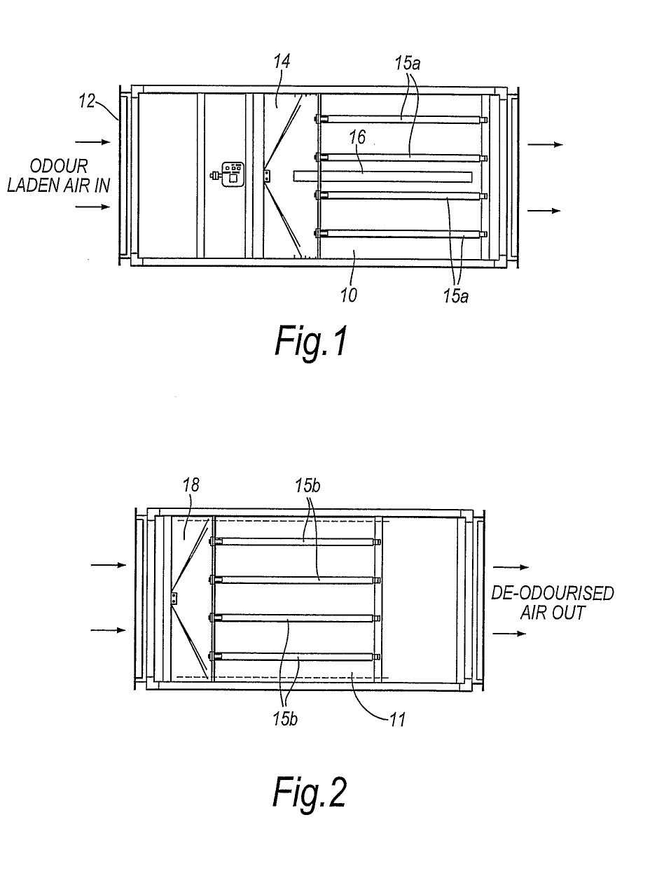

Referring to FIGS. 1 and 2, the apparatus according to one embodiment consists of two sections 10 and 11 respectively. In the first section 10, as shown in FIG. 1, odour laden air is supplied to an inlet 12 and on entry into the section 10 the air is irradiated by ultraviolet light at a wavelength suitable to produce ozone. In this case it has been found that ultraviolet light at a wavelength of 185 nanometers is appropriate. The ultraviolet light is provided by a plurality of UV lamps 15a which are positioned in parallel and equidistant from each other within the first section 10. In this way, the air obtains the same exposure to the ultraviolet radiation.

In order to promote the creation of ozone, the air passing through the inlet 12 is subjected to means for creating a diffuse, turbulent air flow and this is represented by two air turbulators 14. The turbulators form the air into a circular vortex. Additionally, a catalyst is provided within the section 10 to promote the production of ozone. In this embodiment the catalyst is in the form of a titanium dioxide coated metal sheet 16 which is located centrally in the section 10.

Within the section 10, due to the action of the UV light, some of the oxygen (O₂) within the odour laden air stream is broken down into single oxygen atoms. These atoms attach themselves to a complete oxygen (O₂) molecule which then forms ozone (O₃). The ozone thus produced breaks down the odour-forming compounds in the input air stream by oxidation. Similarly, grease molecules in the air are broken down into carbon dioxide and water.

The partially treated air exiting the section 10 is typically passed through ducting towards an outlet to be discharged to the atmosphere. However, before being discharged, the air is passed into the second section 11 where any residual ozone is removed. This is achieved by illuminating the air flowing through the section 11 with ultraviolet light at a suitable wavelength to convert the ozone into single atoms which in turn revert back to complete oxygen molecules. In the present embodiment this is achieved by using UV light at a wavelength of 254 nanometers. As in the first section 10, the ultraviolet light is provided by a plurality of UV lamps 15b which are arranged in a similar configuration to the first section 10. The process in the section 11 is enhanced by lining the section with a highly reflective surface such as may be provided by an aluminium alloy sold under the trade name Alanod.

The air output from the section 11 is odourless and also contains no ozone so it can be safely discharged to atmosphere or into any controlled environmental space. Any carbon dioxide and water formed by breaking down grease molecules can also be discharged through section 11.

If desired, the air leaving the section 10 can be subjected to turbulation prior to entry into section 11 by utilising turbulators 18. Further, baffles which may be either stationary or moveable may be provided within either or both sections 10 and 11 in order to maintain the turbulent flow of air through the sections.

The ultraviolet light can be produced by conventionally available UV lamps and they may be contained within one or more airtight/light tight casings with protective devices to prevent the accidental exposure of personnel to ultraviolet light.

The odour control apparatus described above is an ultraviolet based system that results in complete removal of odorous compounds and grease from the air. The apparatus can be designed as either a section or sections to be mounted within an existing air handling plant or as a free standing, self contained unit complete with its own air moving device.

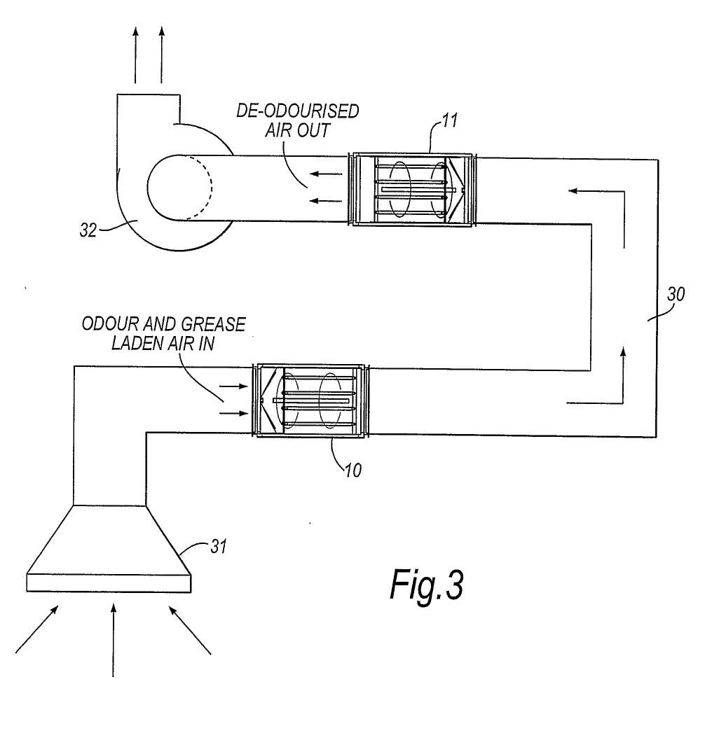

FIG. 3 shows an embodiment of the invention, in which the apparatus described above has been installed as part of a kitchen extraction system. The first section 10 is positioned in the flow of air entering through a kitchen extraction hood 31, and the air laden with odour and grease enters the section 10 and is treated as described above, with reference to FIG. 1.

The apparatus is arranged such that the partially treated air exiting the first section 10 travels along an interconnecting duct section 30 before entering the second section 11. Although the odour removal takes place almost instantaneously in the air passing through the first section 10, the process of breaking down the grease in the air is slower. However, using this embodiment, it has been found that the ozone produced in the first section 10 continues to work on breaking down the grease while the air passes through the interconnecting duct from the first section 10 to the second section 11. This results in a particularly effective reduction in grease, which is also prevented from accumulating on the inside of the duct between the first section 10 and the second section 11.

As the air approaches the outlet to the atmosphere, any remaining ozone is converted to oxygen in the second section 11, as described above with reference to FIG. 2. The carbon dioxide and water produced by breaking down the grease in the first section 10 and the interconnecting duct 30 are discharged to the atmosphere through the second section 11. An extractor fan 32 may be provided as the air moving device for the extractor system.

This embodiment is particularly useful in kitchen extractor applications, where it is necessary to remove grease from the air as well as odours. In such applications, it is necessary to clean the extraction ducts regularly to remove deposited grease, in order to reduce the risk of fire. By implementing the odour removal apparatus of the invention in this way, the odour removal is combined with an effective removal of grease and a significant reduction in the build-up of grease along the extraction duct. This in turn reduces the frequency with which the ducts need to be cleaned, resulting in a maintenance cost saving. It is therefore particularly desirable to employ a large distance between the first section and the second section in this embodiment, in order to maximise the effectiveness of the ozone in breaking down the grease as it passes through the duct, and thereby to increase the amount of the duct which benefits from a reduction in grease build-up.

The first section 10 is positioned close to the inlet, to maximise the effect of the reduction in grease build-up along the duct, and the second section is positioned close to the outlet, in order to provide the maximum time during the passage of air though the extractor system for the grease particles to be removed by the ozone.

In certain air extraction applications, in particular in some commercial kitchen extraction systems, the air at the inlet of the extraction hood is particularly laden with soot particles. It has been found that this can lead to the soot collecting on the UV lamps 15a in the first section 10, reducing their effectiveness in generating ozone and requiring them to be frequently cleaned.

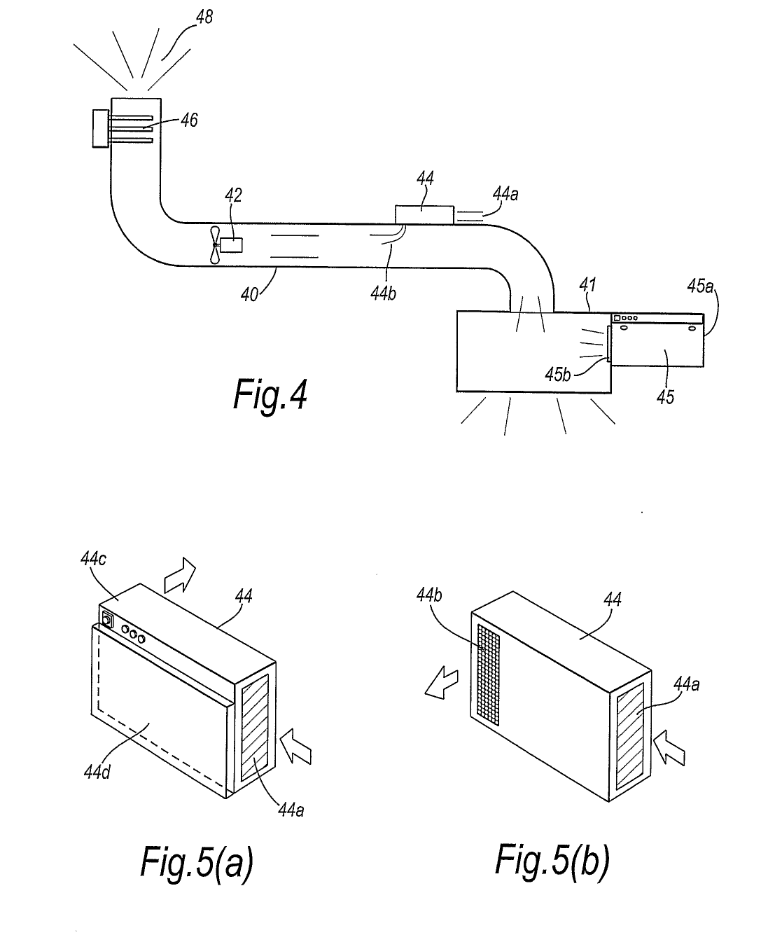

FIG. 4 shows an alternative embodiment of the invention, in which this problem is overcome by using air taken from outside the contaminated air stream to produce the ozone, and injecting the ozone into the contaminated air stream.

The arrangement of FIG. 4 is similar to that shown in FIG. 3, except that an ozone generator 44 is mounted outside the contaminated air stream, instead of in the duct. As in FIG. 3, the arrangement of FIG. 4 is used with a kitchen or industrial collection hood 41, through which the air enters and passes into a duct 40, finally exiting the extraction system by being discharged to atmosphere at the other end 48 of the duct. The extraction hood and duct may be part of an existing extraction system into which the present invention is installed. A system fan 42 is also provided in the duct 40, in order to provide the necessary flow through the extraction system. The extraction fan 32 of FIG. 3 may be used instead of the system fan 42 shown in FIG. 4, and vice versa.

Instead of the air being constrained to pass through a first section 10, as shown in FIG. 3, the air simply passes through the duct, but is injected with ozone produced outside the duct in an ozone generator 44. The ozone generator 44 takes air from the surrounding area at its inlet 44a, outside the duct, and converts some of this air into ozone using UV lamps at a wavelength of 185 nm, as described previously in connection with FIG. 1. The produced ozone is then injected into the contaminated air stream in the duct 40 through an outlet 44b of the ozone generator 44, or is simply drawn into the air stream by the flow through the duct 40.

FIG. 4 shows the ozone generator 44 mounted on the outside of the duct 40, but it may also be mounted in alternative positions, such as that illustrated by ozone generator 45. Ozone generator 45 is identical to ozone generator 44, and is simply intended to illustrate an alternative position for the ozone generator, in this case mounted adjacent to the collection hood 41. It should be appreciated that typically only one of the depicted ozone generators is used, and it is located in one or other of the alternative positions shown.

Where the ozone generator 45 is provided adjacent to the collection hood 41, air is again taken from the surrounding area at an inlet 45a, outside the contaminated air flowing through the duct 40, and the ozone produced in the ozone generator 45 is introduced from an outlet 45b into the air flowing through the hood 41.

As in the arrangement of FIG. 3, any residual ozone contained in the air flowing through the duct 40 close to its outlet is converted back to oxygen by means of UV lamps at a wavelength of 254 nm. FIG. 4 shows a simple UV ‘stab-in’ unit 46 comprising a plurality of UV lamps at a wavelength of 254 nm, which are introduced from the side of the duct in the form of a single removable unit, for convenience. However, an arrangement corresponding to the second section 11 shown in FIG. 3 may be used instead. FIGS. 5(a) and 5(b) show in more detail the ozone generator 44 shown in FIG. 4 (alternatively positioned ozone generator 45 is identical). FIG. 5(a) is a view from the front of the unit, and shows the air inlet 44a, controls 44c typically including an on/off switch and indicator lamps, and a hinged front access panel 44d to facilitate maintenance of the unit. The air inlet 44a provides an entry grille with adjustable regulator to control the flow of air into the unit.

FIG. 5(b) is a view from the rear of the ozone generator, showing the ozone outlet 44b, which comprises the interface of the unit with the duct, in FIG. 4. It will be appreciated that when the ozone generator 44 is mounted on the duct 40, the air will enter through the inlet 44a, pass through the ozone generator to generate ozone, and exit the ozone generator through the outlet 44b, passing through a suitable orifice in the wall of the duct 40 and into the contaminated air stream.

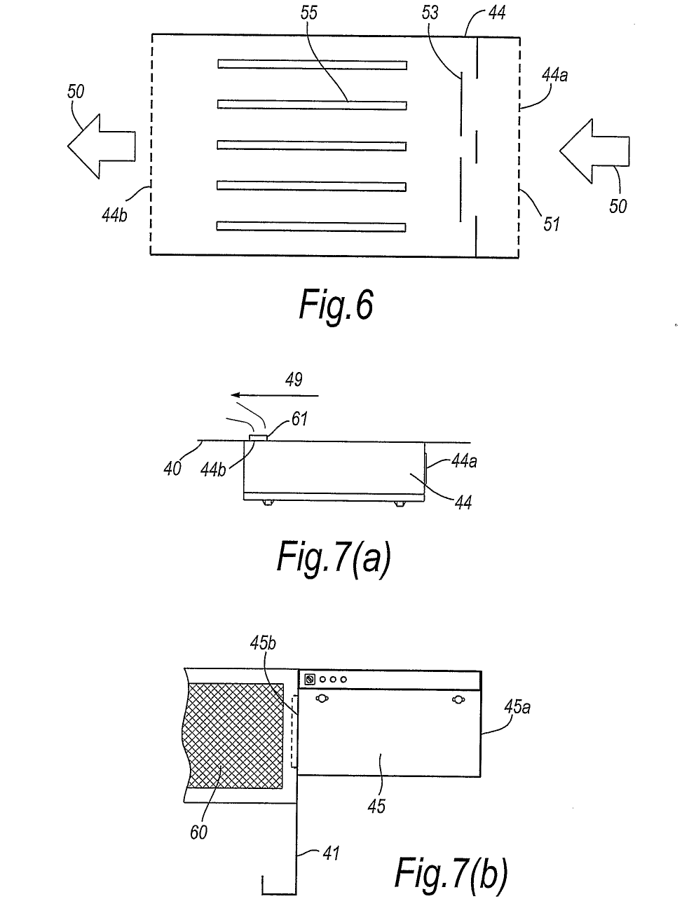

FIG. 6 shows schematically a cross section through the ozone generator 44, in order to illustrate its operating principle. The direction of air flow through the ozone generator 44 is shown by the arrows 50. The air from the surrounding area enters the inlet 44a through an adjustable inlet grille 51. A light blocker 53 is provided in the unit to prevent UV light from spilling out of the inlet, both for safety reasons and to improve the effectiveness of the ozone generation. As can be seen schematically in FIG. 6, the light blocker may comprise a series of overlapping surfaces which are spaced apart in the direction of the air flow, such that there is no direct line of sight between the UV lamps and the inlet, but the air is still able to flow through the light blocker 53. It will be appreciated that any other suitable arrangement of light blocker may be used. The light blocker may also be used to introduce an element of mixing, or turbulence generation, into the air flow, in order to improve the effectiveness of the subsequent ozone generation (see below) by increasing the uniformity of the exposure of the air to the UV light.

After the air has passed into the unit through the inlet grille 51 and the light blocker 53, it is exposed to a plurality of UV lamps 55 at a wavelength of about 184 nm, in order to produce ozone, as described previously. The ozone then passes through the outlet 44b and into the contaminated air stream via a suitable interface with the duct.

FIGS. 7(a) and 7(b) show in more detail the attachment of the ozone generator to the duct 40 or collection hood 41, respectively, of FIG. 4. FIG. 7(a) shows the ozone generator 44 mounted to the underside of the duct 40. The direction of air flow through the duct is shown by arrow 49. The surrounding air outside the duct enters the ozone generator 44 at inlet 44a, and the generated ozone is introduced into the air flow in the duct via outlet 44b. At the interface between the ozone generator 44 and the duct 40, an opening is provided in the duct, with a spigot 61 inserted into it. This allows the ozone to pass from the outlet 44b into the duct 40, while facilitating the mounting of the ozone generator 44 against the duct.

FIG. 7(b) shows the ozone generator 45 mounted in the alternative position shown in FIG. 4, adjacent to an existing collection hood 41 which, in the illustrated example, is provided with a grease filter 60. In this arrangement, the ozone generator 45 takes in air at the inlet 45a, from outside the contaminated air stream, and introduces the produced ozone from the outlet 45b into the hood 41, behind the grease filter 60.

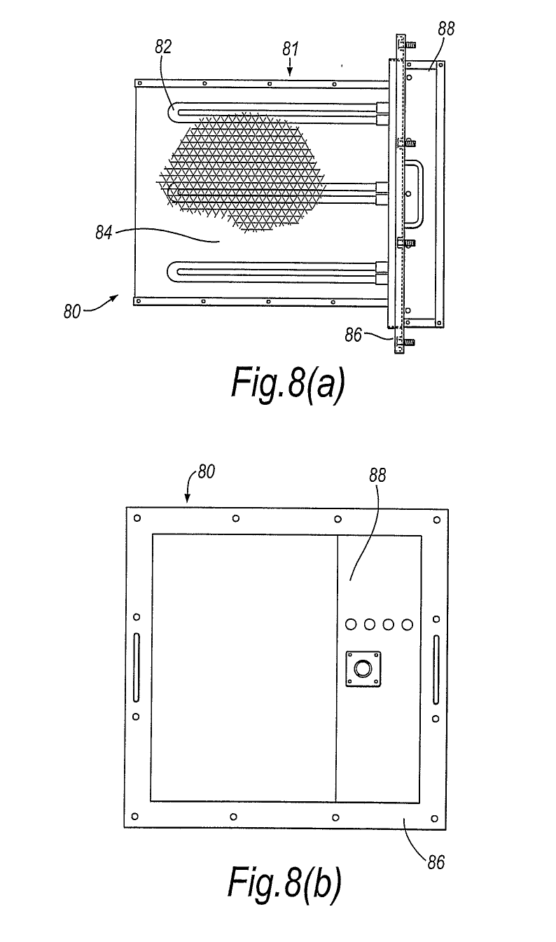

FIGS. 8(a) and 8(b) show the general arrangement of a stab-in duct mounted UV lamp unit 80, which may be removably mounted in the duct in order to provide for ease of maintenance. Such an arrangement may be used to provide the ozone generating section or the residual ozone removing section (see stab-in unit 46 in FIG. 4), depending on the wavelength of the UV lamps.

FIG. 8(a) shows a side view of the unit 80, arranged in this case as an ozone producing unit having three U-shaped UV lamps 82 at a wavelength of 184 nm, which produce ozone from air passing the lamps in the direction of the air flow indicated by arrow 81. A titanium mesh cage 84, shown in partial cross-section, surrounds the UV tubes to act as a catalyst and also to provide protection for the UV tubes. When the unit is installed in the duct, the portion including the UV lamps 82 and the titanium mesh 84 is located in the duct via a suitable recess in the duct wall, and the unit 80 is secured to the duct wall by means of a mounting frame 86, using suitable fastening means. An electrical control box 88, on the other side of the mounting frame 86, remains outside the duct so that the controls of the unit can be accessed without removing it from the duct. FIG. 8(b) shows a plan view of the unit 80, and illustrates how the electrical control box 88 and associated indicators are accessible when the unit 80 is mounted in the duct by means of the mounting frame 86.

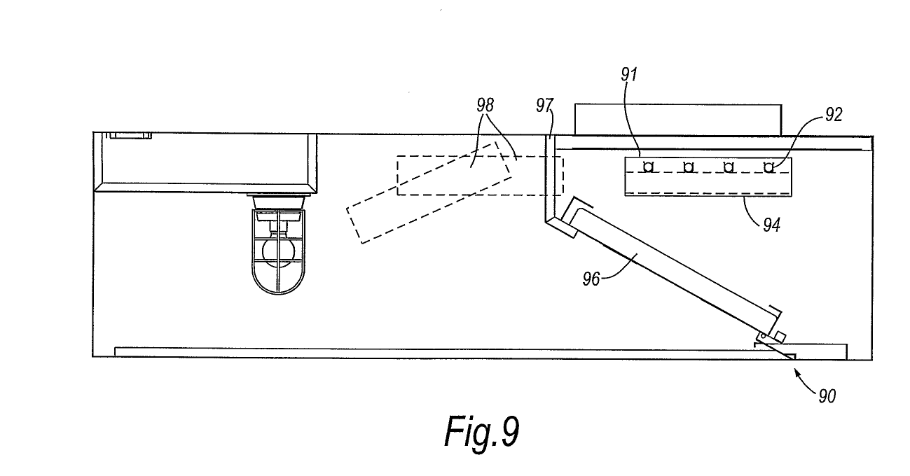

In an alternative arrangement of the invention, an ozone generating UV lamp unit may be installed directly in a kitchen extraction hood, instead of using a hood mounted external ozone generator 45 (as shown in FIGS. 4 and 7(b)) or a duct mounted UV lamp unit 10 (as shown in FIG. 3). FIG. 9 shows a cross-section through an extraction hood 90, in which an ozone generating UV lamp unit 91 is installed.

The UV lamp unit 91 includes UV lamps 92 at a wavelength of about of 184 nm, for producing ozone from the contaminated air stream passing into the extraction hood 90 from below. In accordance with the conventional extraction hood into which the unit is installed, the contaminated air first passes through a conventional grease filter 96, and is then exposed to the UV lamp unit 91, where oxygen in the air is converted to ozone, before continuing into an adjacent duct (not shown) as in the previously described arrangements. As shown in FIG. 9, the UV lamp unit also includes a titanium mesh 94 through which the contaminated air passes before passing the UV lamps 92. The titanium mesh 94 acts both as a catalyst to increase the production of ozone, and also introduces turbulence into the air flow to increase the effectiveness of the exposure of the air to the UV radiation, again improving the production of ozone and providing better mixing of the produced ozone with the contaminants.

In the arrangement of FIG. 9, in order to prevent escape of the UV light, the UV lamp unit 91 is installed in a portion of the extraction hood 90 contained by the arrangement of the grease filter 96 and a removable access panel 97, which provides access to this portion of the hood. The removable access panel 97 is used to install the UV lamp unit in position behind the grease filter 96. Dotted lines 98 show the path taken by the UV lamp unit 91 when being installed into position through the opening provided by removing the access panel 97. The lamp unit 91 is mounted on slides to facilitate easy removal and installation, so that it can be simply positioned directly below a spigot connection with the adjacent duct.

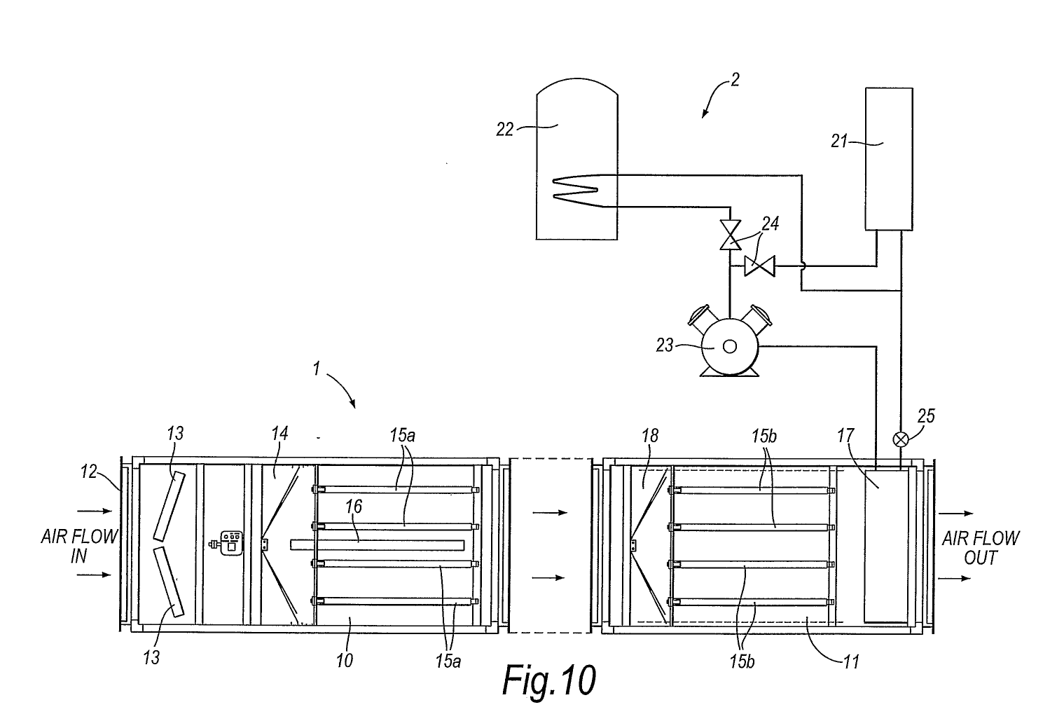

FIG. 10 shows a modification to the present invention whereby the odour removal apparatus is combined with a refrigeration circuit of an energy recovery system to enable heat energy to be recovered from the clean exhausted air after odour removal.

Referring to FIG. 10, the odour removal apparatus 1 is as described above and as shown in FIGS. 1 and 2, and the first and second sections 10, 11 may be separated by an interconnecting duct 30 as shown in FIG. 3. However, the odour removal apparatus is provided with an evaporator in the form of a heat recovery coil 17 which is mounted in the discharge of the odour removal apparatus, in the second section 11.

In addition, a pair of unit air filters 13 are disposed between the inlet 12 and the first section 10. These may be in the form of washable polyester foam or grease filters. The exhaust air from the odour removal system is mechanically cooled by refrigeration. Both sensible and latent energy is removed which in turn is deposited into one side 21 of the recovery system 2 which is an air system to provide space heating.

If predetermined conditions are satisfied then the recovered energy can be deposited into a second side 22 of the recovery system 2 which is a hot water tank to provide domestic hot water to a building.

During periods when both elements of the recovery system are near satisfied then by regulating the flow of refrigerant gas, temperature of the air and water can be regulated so that both air and water can be heated simultaneously.

The energy recovery process employed by the combined system will now be described in more detail by referring to the elements of the energy recovery system 2 shown in FIG. 10.

Vapour compression is employed to provide the cooling effect within the exhaust air and the heating effect in the recovery system 2.

Starting at a compressor 23 discharge where the temperature of a refrigerant gas has been elevated by mechanical compression, the hot gas passes through condensers where heat energy is removed and is passed into either an air 21 or water system 22. Control valves 24 are arranged between the compressor 23 and the air and water system 21, 22 to automatically change priority from air to water if desired. After passing through the air and/or water system 21,22 the high pressure cooled refrigerant liquid passes through an expansion valve 25 through which the fluid pressure is lowered. The low-pressure fluid enters the evaporator 17 of the odour control system where it evaporates by absorbing heat from the exhaust air. The warmed gas re-enters the compressor 23 and the whole cycle is repeated.

A system temperature control unit (not shown) continually monitors the conditions within both of the recovered heat energy systems and the exhaust air from the odour removal system. In this way the most beneficial energy recovery can be achieved.

In addition to the above, mechanical cooling can be provided to the treated space by a system of refrigerant reversing valves, converting the evaporator into a condenser and the condenser into an evaporator.

In this way the combined system will enable odorous compounds to be removed from the air and also enable surplus energy contained within the exhausted air to be recovered and transferred to another medium, for example a ventilation system serving the building or a hot water storage tank.

This process is of particular use when applied for example to a kitchen exhaust system, as the recovered energy will provide economical pre-heating to the hot water system of the kitchen or indeed any area within a building.

It will be appreciated that in the above-described embodiments, the various arrangements for producing ozone and removing residual ozone may be used in any combination, as appropriate, in order to achieve the object of treating the input gas to remove contaminants. Furthermore, the turbulators, catalysts, and reflective lining referred to in connection with the first embodiment may also be used for the same effect, as appropriate, in other embodiments.

What is claimed is:

1. An apparatus for removing unpleasant odours from an input gas comprising:
a first section (10) through which the input gas is constrained to travel and which treats the input gas by exposure to ozone; and
a second section (11) which converts into oxygen any remaining ozone in the gas stream issuing from the first section, wherein the first section is spaced apart from the second section.

2. The apparatus according to claim 1, wherein the first section is spaced from the second section by an interconnecting duct.

3. The apparatus according to claim 1, wherein the first section (10) includes means for producing ozone from air in the input gas.

4. The apparatus according to claim 1, further comprising means (44) for producing ozone from air outside the input gas, and introducing the produced ozone into the input gas in the first section (10).

5. The apparatus according to claim 3, wherein ultra-violet light of a first wavelength is used in order to create the ozone and ultra-violet light at a different wavelength to the first wavelength is utilised to convert ozone to oxygen in the second section.

6. The apparatus according to claim 5, wherein the ultra-violet light in the ozone producing means is at 185 nanometers and the ultra-violet light in the second section is at 254 nanometers wavelength.

7. The apparatus according to claim 5, wherein the ultraviolet light is provided by a plurality of lamps (15a, 15b) which are positioned in parallel to each other and equidistant from each other within the ozone producing means and the second chamber (11), respectively.

8. The apparatus according to claims 3, wherein a catalyst is provided within the ozone producing means to promote the production of ozone.

9. The apparatus according to claim 8, wherein the catalyst is in the form of a titanium dioxide coated metal sheet (16).

10. The apparatus according to claim 1, wherein the second section is lined with a highly reflective surface.

11. The apparatus according to claim 10, wherein the highly reflective surface is an aluminium alloy.

12. The apparatus according to claim 1, further comprising at least one baffle arranged to maintain air flow in the apparatus.

13. The apparatus according to claim 1, further comprising a heat recovery coil (17) mounted adjacent to the second section in order to recover heat energy from the exhaust air.

14. A method of removing unpleasant odours from an input gas, the method comprising the steps of:
providing first gas processing means (10) through which the input gas is constrained to travel and which treats the input gas by means of exposing the input gas to ozone;
providing second gas processing means (11) which converts into oxygen any remaining ozone in the air stream issuing from the first gas processing means; and
locating the first gas processing means at an inlet of a section of duct (30) and the second gas processing means at an outlet of the section of duct so that the first and second gas processing means are spaced apart along the duct.

15. A method according to claim 14, wherein the first gas processing means (10) treats the input gas by producing ozone from air in the input gas.

16. A method according to claim 14, comprising the step of producing ozone from air outside the input gas, and introducing the produced ozone into the input gas in the first gas processing means (10).

17. The apparatus according to claim 4, wherein ultra-violet light of a first wavelength is used in order to create the ozone and ultra-violet light at a different wavelength to the first wavelength is utilised to convert ozone to oxygen in the second section.

18. The apparatus according to claim 17, wherein the ultra-violet light in the ozone producing means is at 185 nanometers and the ultra-violet light in the second section is at 254 nanometers wavelength.

19. The apparatus according to claim 17, wherein the ultraviolet light is provided by a plurality of lamps (15a, 15b) which are positioned in parallel to each other and equidistant from each other within the ozone producing means and the second chamber (11), respectively.

20. The apparatus according to claim 4, wherein a catalyst is provided within the ozone producing means to promote the production of ozone.

21. The apparatus according to claim 20, wherein the catalyst is in the form of a titanium dioxide coated metal sheet (16).
